(19) United States
(12) Patent Application Publication
(10) Pub. No.: US 2014/0205656 A1
(43) Pub. Date: Jul. 24, 2014

(54) MODIFIED CATIONIC LIPOSOME ADJUVANTS

(71) Applicant: Statens Serum Institut, Copenhagen S (DK)

(72) Inventors: Dennis Christensen, Frederiksberg (DK); Karen Smith Korsholm, Hillerod (DK); Else Marie Agger, Copenhagen S (DK); Peter Andersen, Bronshoj (DK)

(73) Assignee: Statens Serum Institut, Copenhagen S (DK)

(21) Appl. No.: 14/162,511

(22) Filed: Jan. 23, 2014

Related U.S. Application Data
(63) Continuation of application No. 13/129,079, filed on Aug. 4, 2011, now abandoned.

(30) Foreign Application Priority Data
Nov. 12, 2008 (DK) PA 2008 01573

(51) Int. Cl. A61K 9/127; A61K 39/12; A61K 39/015; A61K 39/145; A61K 39/04

(52) U.S. Cl. CPC A61K 9/127; A61K 39/145; A61K 39/04; A61K 39/015; A61K 39/12
USPC 424/450; 424/209.1; 424/192.1; 424/272.1; 424/204.1

(57) ABSTRACT

The present invention relates to the use of vaccines with adjuvants comprising cationic liposomes where neutral lipids has been incorporated into the liposomes to change the gel-liquid phase transition and thereby modifying the IgG sub-type response and enhancing the CD8 response of the liposomal adjuvant. This technology can be used to increase the production of IgG2 antibodies. This sub-type of antibodies (IgG2 in mice corresponding to IgG3 in humans) have been shown to selectively engage Fc activatory receptors on the surface of innate immune cells leading to enhanced proinflammatory responses and thereby a more efficient immune response with higher levels of protection in animal models of e.g. malaria and Chlamydia. The use of adjuvants which selectively give rise to higher levels of IgG2 antibodies will improve the effect of vaccines e.g. against intracellular infections. Furthermore the technology can be used to induce a CD8 response which has been reported to improve the effect of vaccines against e.g. HPV, HIV, influenza and cancer have been shown to selectively engage Fc activatory receptors on the surface of innate immune cells leading to enhanced proinflammatory responses and thereby a more efficient immune response with higher levels of protection in animal models of e.g. malaria and Chlamydia. The use of adjuvants which selectively give rise to higher levels of IgG2 antibodies will improve the effect of vaccines e.g. against intracellular infections. Furthermore the technology can be used to induce a CDS response which has been reported to improve the effect of vaccines against e.g. HPV, HIV, influenza and cancer.

### CROSS-REFERENCE TO RELATED APPLICATIONS

This application is a continuation of U.S. patent application Ser. No. 13/129,079, filed May 12, 2011 (35 USC 371 completion on Aug. 4, 2011), which is a national stage of International Patent Application No. PCT/DK2009/000233, filed Nov. 10, 2009, which applications are incorporated by reference herein in their entireties.

### FIELD OF INVENTION

The present invention discloses methods for modifying the IgG sub-type response and enhancing the CD8+ T cell response of adjuvants comprising cationic liposomes by incorporating neutral lipids e.g. phospholipids that modifies the gel-liquid crystalline phase transition (Tm) of the liposome, adjuvants and vaccines.

### BACKGROUND OF THE INVENTION

The majority of novel generation vaccines are based on highly pure proteins or peptides derived from the pathogen, however due to the inherently low immunogenicity of proteins and peptides major focus has been directed towards design of adjuvants that serve to enhance the immune response of the vaccine. Although a number of new adjuvant systems have been identified during the past 20-30 years, the need for new adjuvant systems is still recognized (Moingeon, Haensler et al. 2001) which is evident in the paucity of choices available for clinical use.

An adjuvant (from latin adjuvare, to help) can be defined as any substance that when administered in the vaccine serves to direct, accelerate, prolong and/or enhance the specific immune response. Depending on the nature of the adjuvant it can promote a cell-mediated immune response, a humoral immune response or a mixture of the two. When used as a vaccine adjuvant an antigenic component is added to the adjuvant. Since the enhancement of the immune response mediated by adjuvants is non-specific, it is well understood in the field that the same adjuvant can be used with different antigens to promote responses against different targets e.g. with an antigen from M. tuberculosis to promote immunity against M tuberculosis or with an antigen derived from a tumor, to promote immunity against tumors of that specific kind.

Presently, only a few adjuvants are accepted for human use e.g. aluminum-based adjuvants (AlOH-salts) and MF-59. Both of these adjuvants are inducers of a humoral immune response but provide only negligible cell-mediated immunity (CMI). As the generation of a robust CMI response is considered essential e.g. for a protective immune response against many intracellular pathogens like M tuberculosis or for the eradication of tumors, there has been an intensive search for more potent adjuvant formulations for inclusion in new vaccines.

In addition, many of the remaining disease targets for which there is presently no effective vaccines rely on varying levels of CMI responses with or without an associated humoral response. HIV and Chlamydia both belong to this category of global health problems that are crucially dependent on a mixed CMI and humoral response for protection but also many of the existing vaccines may benefit from an improved adjuvant technology that would stimulate both arms of the immune system. This is illustrated by influenza where antibodies neutralize the infectivity of the virus and the cytotoxic T-cells reduce viral spread and thereby serve to enhance the recovery from influenza (McMichael, Gotch et al. 1981).

Dimethyldioctadecylammonium bromide, -chloride, -phosphate, -acetate or other organic or inorganic salts (DDA) is a lipophilic quaternary ammonium compound, which forms cationic liposomes in aqueous solutions at temperatures above ˜40° C. DDA has been used extensively as an adjuvant (see Hilgers for a review). In e.g. administration of Arquad 2HT, which comprises DDA, in humans was promising and did not induce apparent side effects (Stanfield, Gall et al. 1973). The combination of DDA and immunomodulators as adjuvants have been described e.g. DDA and TDB, DDA and MMG or DDA and MPL which all showed a very clear synergy enhancing the immune response compared to the responses obtained with either DDA alone or the immunomodulator alone. DDA-based formulations are therefore promising adjuvants candidates for inclusion in vaccines. The combination of cationic liposomes (e.g. DDA) and a non-ionic surfactant has been used in an oil emulsion delivering drugs to cells (Liu, Liu et al. 1997), furthermore cationic amphiphiles and non-ionic surfactants have been used separately to form mixtures of cationic liposomes and neutralliposomes to target tumor cells with greater efficiency compared to cationic liposomes alone (Campbell, Brown et al. 2002).

Recently, it has become evident that antibodies not only neutralize e.g. virus but can also regulate immune response through interacting with Fe receptors on the surface of innate immune cells. In particular, the IgG2 subclasses in mice have been associated with the most potent proinflammatory and effective antibody response. Hence, vaccine-induced IgG2 was found particular effective at mediating immunity to blood stage malaria infection in mouse models (Ahlborg, Ling et al. 2000). Although it is not possible to identify a human analogue, IgG3 shares many characteristics with mouse IgG2 including a more effective anti-malaria response. In epidemiological studies carried out in high endemic areas, the level of IgG3 has been shown to correlate with resistance against the development of clinical malaria (Taylor, Allen et al. 1998). The higher activity of IgG2 has also attracted a lot of interest in other fields including chlamydia where this isotype is found responsible for antibody enhancement of Th1 activation and the subsequent protection (Moore, Ekworomadu et al. 2003). Over the last 5 years, there has been a breakthrough in our understanding of how the various antibody isotypes interact with either activatory or inhibitory Fc receptors and thereby mediate the differential activity observed in vivo (Nimmerjahn, Bruhns et al. 2005). Thus, IgG1 antibodies selectively bind to inhibitory FcγRIIB expressed on dendritic cells whereas IgG2 antibodies preferentially engage the activatory Fcγ:RIV receptor crucial for the higher in vivo activity observed as e.g. enhanced phagocytosis and release of inflammatory mediators (Regnault, Lankar et al. 1999). The discovery of inhibitory receptors and how these interacts with antibodies will also make this possible to generate antibodies or therapeutic tumor vaccines with improved activity e.g. by using immune complexes that selectively engage activatory receptors (Nimmerjahn 2007 cur.op.imm.).

There is therefore a growing interest for the quality of the vaccine-induced antibody response which has crucial importance for the development of the cellular immune response and thereby the protective or therapeutic properties of the vaccine. An adjuvant that selectively induces a high amount of antibodies that engage activatory receptors will therefore be very valuable in this context.

From immunogenicity studies in mice, it is known that the combination of DDA/TDB as an adjuvant induces a strong Th1 type of immune response characterized by substantial production of IFN-γ, and at the same time levels of IgG1 comparable to what is seen using conventional aluminum hydroxide (alum) (Davidsen, Rosenkrands et al. 2005). In addition, DDA/TDB in combination with the mycobacterial vaccine antigen Ag85B-ESAT-6 gave rise to high titers of IgG2b, however although the levels were clearly above that seen in the alum group the level was considerably still lower compared to what was seen when analysing IgG1 titers. Other studies have also shown that both neutral and cationic liposomes systems can induce/increase both IgG1 and IgG2 responses (Philips et al. 1992; Philips et al, 1996, WO2004/110496, WO2006/002642) and that the general levels of the different IgG subtypes can be increased by using solid state liposomes instead of liquid state liposomes (Ivanoff et al. 1996 Gregozewska et al. 2003). But none of these addresses how to selectively increase the amount of IgG2 and at the same time maintaining or reducing the level of IgG1. Improvement of this IgG2 inducing effect is therefore much needed.

Cytotoxic CD8+ T cells have the capacity to directly kill an infected cell, and as such they are potent effectors against many diseases. Inducing CD8+ T cell responses by vaccination has great implications for prophylactic vaccines and therapies for viral infections and cancers but also against pathogens multiplying in intracellular vesicles where antigen is cross-presented on MHC class I. Common vaccine strategies to achieve CD8+ T cells responses include the use of viral vectors, DNA immunization and co-injecting peptides and cytokines, which have drawbacks when it comes to repeated immunizations (viral vectors), efficacy (DNA vaccination) and systemic effects (cytokines).

We have observed that incorporation of a neutral lipid (DSPC) in DDA/TDB liposomes can induce a CD8+ T cell response. When combined with the Human Papilloma Virus 16 (HPV-16) antigen E7, the DDA/TDB/DSCP liposomes primed antigen specific CD8+ T cells that produced Interferon gamma (IFNg) and tumor necrosis factor alpha (TNFa) upon antigen restimulation (FIG. 8). Furthermore, this immune response was able to significantly reduce tumor size in a mouse model of HPV induced cancer (FIG. 9). This has not been observed with the DDA/TDB liposomes.

### SUMMARY OF THE INVENTION

The present invention discloses the use of neutral lipids e.g. phospholipids to enhance the CD8+ T cell response and modify the immunoglobulin iso-/subtype response of cationic liposomes also comprising an immunomodulator as an adjuvant, the adjuvant and a vaccine comprising this adjuvant. Using vaccine adjuvants where lipids has been incorporated in cationic liposomes to increase the gel-to-liquid phase transition temperature and thereby selectively increase the amount of IgG2 and at the same time maintain or reduce the level of IgG1, thereby improving the effect of vaccines against particularly intracellular infections, e.g. tuberculosis (TB), malaria, chlamydia, influenza and Human Immunodeficiency Virus (HIV), cancers and infectious diseases causing cancers e.g. Human Papilloma Virus (HPV).

### BRIEF DESCRIPTION OF THE DRAWINGS

FIG. 1. Differential scanning heat capacity curves for DDA/DSPC/TDB liposomes with different DDA:DSPC ratios according to the figure. The curves have been normalized to molar content. Notice that the scans have been displaced on the heat capacity axis for clarity.

FIG. 2. Zeta-potentials of DDA/DSPC/TDB liposomes with different DDA:DSPC ratios according to the figure. The formulations were diluted 300 times prior to measurement.

FIG. 3. BALB/c mice (n=4) were vaccinated s.c. with 1 μg of influenza split vaccine either without adjuvant (▪) or in combination with DDA/TDB (▴), DDA/DSPC/TDB (▾) or alum (♦). In addition, naive un-vaccinated animals were included (). Four weeks after a single immunization, the presence of influenza vaccine-specific antibodies (IgG1 and IgG2a) was assessed in the sera using ELISA.

FIG. 4. C57BL/6 mice (n=4) were vaccinated three times s.c. (two weeks interval between each immunization) with 2 μg of Ag85B-ESAT-6 in combination with DDA/TDB (▴), DDA/DSPC/TDB (▾) or alum (♦). In addition, naive un-vaccinated animals were included (). Three weeks after the last immunization, the presence of Ag85B-ESAT-6-specific antibodies (IgG1, IgG2b and IgG2c) was assessed in the sera using ELISA.

FIG. 5. C57BL/6 mice (n=4) were vaccinated three times s.c. (two weeks interval between each immunization) with 10 μg of MSP1-19 in combination with DDA/TDB (▴) or DDA/DSPC/TDB (▾). In addition, naïve un-vaccinated animals were included (). Three weeks after the last immunization, the presence of MSP 1-19-specific antibodies (IgG1, IgG2b and IgG2c) was assessed in the sera using ELISA.

FIG. 6. A) BALB/C mice, B) C57BL/6, C) BALB/c×C57BL/6 F₁ (n=6) were vaccinated three times s.c. (two weeks interval between each immunization) with 5 μg of CtHl in combination with DDA/TDB (▴) or DDA/DSPC/TDB (▾). In addition, naïve un-vaccinated animals were included (). Three weeks after the last immunization, the presence of CtH1-specific antibodies (IgG1 and IgG2a or IgG2c) was assessed in the sera using ELISA.

FIG. 7. C57BL/6 mice (n=4) were vaccinated three times s.c. (two weeks interval between each immunization) with 2 μg of Ag85B-ESAT-6 in combination with DDA/TDB, DDA/D(C18)PC/TDB (D(C18)PC=DSPC), DDA/D(C22)PC/TDB or DDA/D(C18)PC/TDB. Three weeks after the last immunization, the presence of Ag85B-ESAT-6-specific antibodies (IgG1, IgG2c) was assessed in the sera using ELISA. FIG. 8. C57BL/6 mice (n=5) were vaccinated at days 4, 7, 10 and 24 relative to the day of tumor challenge with 5 μg of recombinant E7 in combination with DDA/D(C18)PC/TDB (D(C18)PC=DSPC). A mock vaccine composed of saline mixed with DDA/D(C18)PC/TDB was included. At day eighteen relative to the day of tumor challenge—eight days after third vaccination—mice were bled by periorbital puncture, and pooled PBMCS were analyzed by flow cytometry for cytokine (IFNγ, TNFα) production upon restimulation with recombinant E7 (5 μg/ml).

FIG. 9. C57BL/6 mice (n=5) were injected intradermally with 10̂5 TC-1 tumor cells (expressing the HPV-16 antigen E7). At days 4, 7, 10 and 24 relative to tumor challenge, mice were vaccinated with 5 μg E7 combined with the DDA/DSPC/TDB) adjuvant. A mock vaccine composed of saline mixed with DDA/DSPC/TDB was included. Tumor size was measured twice weekly, and mice with tumors reaching 200 mm² were euthanized. * P<0.05, unpaired t-test.

FIG. 10. Comparison of the particle size distribution (A), long term particle size stability (B) and zeta potential (C) of liposomes comprising DDA/TDB (w/w ratio: 5:1) and DDA/DSPC/TDB (w/w ratio 3:2:1). Data were generated using Dynamic light scattering. Liposomes comprising a neutral lipid increasing the gel-liquid phase transition temperature also increased the average particle size, whereas there was no difference in the observed surface charge. There were no difference in the obtained polydispersity index (DDA/TDB=0.41 and DDA/DSPC/TDB=0.38).

### DETAILED DESCRIPTION OF THE INVENTION

The present invention discloses methods for modifying the IgG sub-type response and enhancing the CD8+ T cell response of adjuvants comprising cationic liposomes by incorporating neutral lipids e.g. phospholipids that modifies the gel-liquid crystalline phase transition (Tm) of the liposome.

The cationic liposome is preferably chosen among dimethyldidodecanoylammonium, dimethylditetradecylammonium, dimethyldihexadecylammonium, dimethyldioctadecylammoniumbromide, -chloride or other organic or inorganic salts hereof (DDA-B, DDA-C or DDA-X commonly abbreviated as DDA), 1,2-dioleoyl-3-trimethylammonium propane (DOTAP), 1,2-dimyristoyl-3-trimethylammonium-propane, 1,2-dipalmitoyl-3-trimethylammonium-propane, 1,2-distearoyl-3-trimethylammonium-propane, 1,2-distearoyl-3-trimethylammonium-propane and dioleoyl-3-dimethylammonium propane (DODAP), N-[1-(2,3-dioleyloxy)propyl]-N,N,N-trimethylammonium (DOTMA). Other types of preferred cationic lipids used in this invention include but are not limited to 1,2-dimyristoyl-3-trimethylammonium-propane (DMTAP), 1,2-dipalmitoyl-3-trimethylammonium-propane (DPTAP) and 1,2-distearoyl-3-trimethylammonium-propane (DSTAP). The cationic liposomes can be stabilized by incorporating glycolipids e.g. with trehalose 6′6′-dibehenate (TDB) or monomycolyl glycerol (MMG).

Preferred types of neutral lipids used in this invention to modify Tₘ consist of phosphatidylcholine (PC), phosphatidylethanolamine (PE), phosphatidylserine (PS) and/or phosphatidylglycerol (PG) containing one or two long chain fatty acids. One particular preferred type of lipid used in this invention to modify Tₘ is 1-Acyl-2-Acyl-sn-Glycero-3-Phosphocholine (DxPC) wherein 1-Acyl and 2-Acyl independently each is a long chain fatty acid containing from 12 to 24 carbon (C) atoms. Examples of such fatty acids are lauric (12C), myristic (14C), palmitic (16C), stearic (18C), arachidonic (20C), behenic (22C) or lignoceric (24C) acid. However, also other C12-C24 hydrocarbon groups are possible because even though the 1-acyl and 2-acyl groups preferably are saturated with no branched side chains they may in minor degree be branched having e.g. methyl and ethyl side chains. 1-acyl and 2-acyl may also have a minor degree of unsaturation, e.g. containing 1-3 double bonds each.

The weight ratio between the cationic lipids and the neutral lipids is preferably between 19:1 (5% neutral lipid) and 4:16 (80% neutral lipid) and most preferably 12:8 (40% neutral lipid).

The present invention also discloses adjuvants modified by above mentioned methods.

The adjuvant can additionally comprise an immunomodulator. The immunomodulator is preferably selected from the group of so-called pathogen-associated molecular patterns (PAMPs) which comprises e.g. TLR-ligands (e.g. MPL (monophosphoryl lipid A) or derivatives thereof, polyinosinic polycytidylic acid (poly-IC) or derivatives thereof, flagellin, CpG, Resiquimod, Imiquimod, Gardiquimod), nucleotide-binding oligomerization domain NOD-like receptors e.g. muramyldipeptide, C-type lectins e.g. the Dectin-1 ligand Zymosan and ligands for the RIG-like receptors. The immunomodulator can also be selected from the group of pathogen-associated molecular patterns for which no receptor has been identified yet e.g. TDM or derivatives thereof (e.g. TDB), MMG or derivatives thereof (PCT/DK2008/000239 (WO 2009/003474) which is hereby incorporated by reference), zymosan, tamoxifen, CpG oligodeoxynucleotides, double-stranded RNA (dsRNA), or ligands for other pathogen-pattern recognition receptors such as muramyl dipeptide (MOP) or analogs thereof.

The present invention further discloses vaccines comprising the adjuvants modified by above-mentioned methods. The vaccine comprises an antigenic component e.g. against tuberculosis, malaria, Chlamydia, influenza, HPV or HIV.

### DEFINITIONS

An adjuvant is defined as a substance that non-specifically enhances the immune response to an antigen. Depending on the nature of the adjuvant it can promote a cell-mediated immune response, a humoral immune response or a mixture of the two. When used as a vaccine adjuvant an antigenic component is added to the adjuvant solution possibly together with other immunomodulators e.g. TLR ligands such as MPL (monophosphoryllipid A) or derivatives thereof, polyinosinic polycytidylic acid (poly-IC) or derivatives thereof, TDM or derivatives thereof e.g. TDB, MMG or derivatives thereof, zymosan, tamoxifen, CpG oligodeoxynucleotides, double-stranded RNA (dsRNA), or ligands for other pathogen-pattern recognition receptors such as muramyl dipeptide (MDP) or analogs thereof. The addition of such TLR ligands can lead to highly accelerated responses of the adjuvant e.g. as shown when combining DDA/TDB with poly-IC (WO 2006/002642). Also, the addition of TLRs may lead to a significant CD8 T cell response as shown for the model antigen ovalbumin (Zaks, Jordan et al. 2006).

Immunomodulators target distinct cells or receptors e.g. toll-like receptors on the surface of APCs. Delivery systems such as the cationic liposomes and immunomodulators can be used together as adjuvants. In addition to being a component in a vaccine, immunomodulators can be administered without antigen(s). By this approach it is possible to activate the immune system locally e.g. seen as maturation of antigen-presenting cells, cytokine production which is important for anti-tumor and anti-viral activity. Thus, the administration of immunomodulators may e.g. support in the eradication of cancer and skin diseases. Examples of immunomodulators which can be administered locally e.g. on the skin, are Taxanes e.g. Taxol, the toll-like receptor 7/8 ligand Resiquimod, Imiquimod, Gardiquimod.

Liposomes (or lipid vesicles) are aqueous compartments enclosed by a lipid bilayer. The liposomes act as carriers of the antigen (either within the vesicles or attached onto the surface) and may form a depot at the site of inoculation allowing slow, continuous release of antigen (Gluck 1995). The lipid components are usually phospholipids or other amphiphiles such as surfactants, often supplemented with cholesterol and other charged lipids. Liposomes are able to entrap water- and lipid-soluble compounds thus allowing the liposome to act as a carrier. Liposomes have been used as delivery systems in pharmacology and medicine such as immunoadjuvants, treatment of infectious diseases and inflammations, cancer therapy, and gene therapy (Gregoriadis 1995). Factors which may have an influence on the adjuvant effect of the liposomes are liposomal size, lipid composition, and surface charge. Furthermore, antigen location (e.g., whether it is adsorbed or covalently coupled to the liposome surface or encapsulated in liposomal aqueous compartments) may also be important.

Cationic liposomes contain lipids which gives the liposome surface a net positive charge. These lipids could be any amphiphilic lipid, including synthetic lipids and lipid analogs, having hydrophobic and polar head group moieties, a net positive charge at physiological pH, and which by itself can form spontaneously into bilayer vesicles or micelles in water.

One particular preferred type of cationic lipids are quaternary ammonium compounds having the general formula NR¹R²R³R⁴—X wherein R¹ and R² independently each is a short chain alkyl group containing from 1 to 3 carbon atoms, R³ is independently hydrogen or a methyl or an alkyl group containing from 12 to 20 carbon atoms, preferably 14 to 18 carbon atoms, and R⁴ is independently a hydrocarbon group containing from 12 to 20 carbon atoms, preferably from 14 to 18 carbon atoms. X is a pharmaceutically acceptable anion, which itself is nontoxic. Examples of such anions are halide anions, chloride, bromide and iodine. Inorganic anions such as sulfate and phosphate or organic anions derived from simple organic acids such as acetic acid may also be used. The R¹ and R² groups can be methyl, ethyl, propyl and isopropyl, whereas R³ can be hydrogen, methyl or dodecyl, tridecyl, tetradecyl, pentadecyl, hexadecyl, heptadecyl, octadecyl nonadecyl and eicocyl groups and R⁴ can be dodecyl, tridecyl, tetradecyl, pentadecyl, hexadecyl, heptadecyl, octadecyl, nonadecyl and eicocyl groups. However, also other C₁₂-C₂₀ hydrocarbon groups are possible because even though the R³ and R⁴ groups preferably are saturated with no branched side chains they may in minor degree be branched having e.g. methyl and ethyl side chains. R³ and R⁴ may also have a minor degree of unsaturation, e.g. containing 1-3 double bonds each, but preferably they are saturated alkyl groups. The cationic lipid is most preferably dimethyldioctadecylammoniumbromide, -chloride or other organic or inorganic salts hereof (DDA), dimethyldioctadecenylammonium chloride, -bromide or other organic or inorganic salts hereof (DODA), or 1,2-dioleoyl-3-trimethylammonium propane (DOTAP), 1,2-dimyristoyl-3-trimethylammonium-propane, 1,2-dipalmitoyl-3-trimethylammonium-propane, 1,2-distearoyl-3-trimethylammonium-propane and dioleoyl-3-dimethylammonium propane (DODAP) and N-[1-(2,3-dioleyloxy)propyl]-N,N,N-trimethylammonium (DOTMA). Other types of preferred cationic lipids used in this invention include but are not limited to 1,2-dimyristoyl-3-trimethylammonium-propane (DMTAP), 1,2-dipalmitoyl-3-trimethylammonium-propane (DPTAP) and 1,2-distearoyl-3-trimethylammonium-propane (DSTAP). They have the ability to form lipid aggregates such as lipid bilayers, liposomes of all types both unilamellar and multilamellar, micelles and the like when dispersed in aqueous medium. The lipid membranes of these structures provide an excellent matrix for the inclusion of other amphiphilic compounds such as glycolipids e.g. MMG or alpha,alpha′-trehalose 6,6′-dibehenate (TDB) which are shown to stabilize vesicle dispersions (Davidsen, Rosenkrands et al. 2006).

A glycolipid is defined as any compound containing one or more monosaccharide or glycerol residues bound by a glycosidic linkage to a hydrophobic moiety such as a long chain fatty acid, a sphingoid, a ceramide or a prenyl phosphate. The glycolipids of this invention can be of synthetic, plant or microbial origin e.g. from mycobacteria. A comprehensive description of glycolipids is described in WO 2006/002642 which is hereby incorporated as reference. The liposomes of this invention can be made by a variety of methods well known in the art (Davidsen, Rosenkrands et al. 2006). The incorporation of the glycolipids TDB or MMG into liposomes/delivery systems which stabilizes the liposomes can be made by a variety of methods well known in the art including simple mixing of liposomes and glycolipids (Davidsen, Rosenkrands et al. 2006).

Neutral liposomes are most often phospholipids such as phosphatidylcholine (PC), phosphatidylethanolamine (PE), phosphatidylserine (PS) and phosphatidylglycerol (PG) containing one or two long chain fatty acids. One particular preferred type of phospholipid used in this invention to modify Tₘ is 1-Acyl-2-Acyl-sn-Glycero-3-Phosphocholine (DxPC) wherein 1-Acyl and 2-Acyl independently each is a long chain fatty acid containing from 12 to 24 carbon (C) atoms. Examples of such fatty acids are lauric (12C), myristic (14C), palmitic (16C), stearic (18C) (DSPC), arachidonic (20C), Behenic (22C) or lignoceric (24C) acid. However, also other C12-C24 hydrocarbon groups are possible because even though the 1-acyl and 2-acyl groups preferably are saturated with no branched side chains they may in minor degree be branched having e.g. methyl and ethyl side chains. 1-acyl and 2-acyl may also have a minor degree of unsaturation, e.g. containing 1-3 double bonds each.

The invention further discloses a vaccine for parenteral, oral or mucosal administration or a delivery system comprising the adjuvant. A preferred vaccine comprises a whole interactivated pathogen e.g. like the currently used influenza split vaccine or an antigenic epitope from an intracellular pathogen e.g. a virulent mycobacterium (e.g. the fusion products Ag85b_TB10.4, Ag85b_ESAT-6_Rv2660, Ag85b_TB10.4_Rv2660 and Ag85a_TB10.4_Rv2660), Plasmodium falciparum (Msp1, Msp2, Msp3, Amal, GLURP, LSAT, LSA3 or CSP), Chlamydia trachomatis (e.g. CT184, CT521, CT443, CT520, CT521, CT375, CT583, CT603, CT610 or CT681), HIV, influenza or Hepatitis B or C. The adjuvant or delivery system can also be used in vaccines for treating cancer, allergy or autoimmune diseases.

The antigenic component or substance can be a polypeptide or a part of the polypeptide, which elicits an immune response in an animal or a human being, and/or in a biological sample determined by any of the biological assays described herein. Alternatively, the antigenic component can be a single peptide, a mixture of different peptides, or a mixture consisting of adjacent overlapping peptides spanning the whole amino acid sequence of a protein. The immunogenic portion of a polypeptide may be a T-cell epitope or a B-cell epitope. In order to identify relevant T-cell epitopes which are recognized during an immune response, it is possible to use a “brute force” method: Since T-cell epitopes are linear, deletion mutants of the polypeptide will, if constructed systematically, reveal what regions of the polypeptide are essential in immune recognition, e.g. by subjecting these deletion mutants e.g. to the IFN-gamma assay described herein. Another method utilizes overlapping oligopeptides (preferably synthetic having a length of e.g. 20 amino acid residues) derived from the polypeptide which can induce a subdominant immune response. Subdominant epitopes and the use of these for vaccination is described in PCT/DK2007/000312 (WO 2008/000261) which is hereby incorporated by reference. The peptides can be tested in biological assays (e.g. the IFN-gamma assay as described herein) and some of these will give a positive response (and thereby be immunogenic) as evidence for the presence of a T cell epitope in the peptide. Linear B-cell epitopes can be determined by analyzing the B cell recognition to overlapping peptides covering the polypeptide of interest as e.g. described in Harboe et al, 1998 (Harboe, Malin et al. 1998).

Although the minimum length of a T-cell epitope has been shown to be at least 6 amino acids, it is normal that such epitopes are constituted of longer stretches of amino acids. Hence, it is preferred that the polypeptide fragment of the invention has a length of at least 7 amino acid residues, such as at least 8, at least 9, at least 10, at least 12, at least 14, at least 16, at least 18, at least 20, at least 22, at least 24, and at least 30 amino acid residues. Hence, in important embodiments of the inventive method, it is preferred that the polypeptide fragment has a length of at most 50 amino acid residues, such as at most 40, 35, 30, 25, and 20 amino acid residues. It is expected that the peptides having a length of between 10 and 20 amino acid residues will prove to be most efficient as diagnostic tools, and therefore especially preferred lengths of the polypeptide fragment used in the inventive method are 18, such as 15, 14, 13, 12 and even 11 amino acids.

A vaccine is defined as a suspension of dead, attenuated, or otherwise modified microorganisms (bacteria, viruses, or rickettsiae) or parts thereof for inoculation to produce immunity to a disease. The vaccine can be administered either prophylactic to prevent disease or as a therapeutic vaccine to combat already existing diseases such as cancer or latent infectious diseases but also in connection with allergy and autoimmune diseases. The vaccine can be emulsified in a suitable adjuvant for potentiating the immune response.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 1000 μg, such as in the range from about 1 μg to 300 μg, and especially in the range from about 1 μg to 50 μg. Suitable regimens for initial administration and booster shots are also variable but are typified by an initial administration followed by subsequent inoculations or other administrations.

The manner of application may be varied widely. Any of the conventional methods for administration of a vaccine are applicable. These are believed to include oral or mucosal application on a solid physiologically acceptable base or in a physiologically acceptable dispersion, parenterally, by injection or the like. The dosage of the vaccine will depend on the route of administration and will vary according to the age of the person to be vaccinated and, to a lesser degree, the size of the person to be vaccinated.

The vaccines are conventionally administered parenterally, by injection, for example, either subcutaneously or intramuscularly. Additional routes of administration include the oral, transcutane, nasal, pulmonary, vaginal and rectal routes. For suppositories, traditional binders and carriers may include, for example, polyalkalene glycols or triglycerides; such suppositories may be formed from mixtures containing the active ingredient in the range of 0.5% to 10%, preferably 1-2%. Liquid formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95% of active ingredient, preferably 25-70%.

The vaccine of choice can e.g. be:
Protein Vaccine: A vaccine composition comprising a polypeptide (or at least one immunogenic portion thereof) a peptide mixture or fusion polypeptide.
Influenza Vaccines: The currently available vaccines are subvirion preparations made from inactivated, detergent-split influenza virus (so-called split vaccines), whole inactivated viruses or recombinant subunit vaccines e.g. containing recombinant haemagglutinin and neuraminidase proteins produced in cell culture with a baculo virus vector. In addition hereto, several novel methods are under development including DNA vaccines, fusion of selected proteins (e.g. the M2 protein) into hepatitis B core antigen, or peptide-based vaccines.
Live recombinant vaccines: Expression of the relevant antigen in a vaccine in a non-pathogenic microorganism or virus. Well-known examples of such microorganisms are Mycobacterium bovis BCG, Salmonella and Pseudomonas and examples of viruses are Vaccinia Virus and Adenovirus.

This invention discloses a method to turn the balance of the IgG response of the liposome/glycolipid adjuvant e.g. DDA/TDB, towards a higher IgG2 (IgG2ₘₒᵤₛₑ=IgG3ₕᵤₘₐₙ) response by modifying the lipid composition and thereby affecting gel-liquid crystalline phase transition of the liposomes. The gel-liquid crystalline phase transition temperature (Tm) has earlier been connected with the ability of liposomes to generate an immune response. This has been shown in numerous studies including e.g. the use of aliphatic nitrogenous bases including DDA (Gall 1966) and the use of 1,2-diacyl-sn-Glycero-3-Phosphocholine (DxPC) (Bakouche and Gerlier 1986). These studies showed that the mean antibody titer was enhanced with increased acyl chain length and on saturation hereof but none have shown that the response can be skewed towards an IgG2 response.

The present invention discloses methods for modifying the IgG sub-type response of adjuvants comprising cationic liposomes e.g. DDA/TDB by incorporating neutral lipids e.g. phospholipids that modifies the gel-liquid crystalline phase transition (Tₘ) of the liposome. A preferred adjuvant disclosed by the invention is an adjuvant comprising liposomes consisting of an immunomodulator, cationic lipids and a neutral phospholipid changing the overall Tₘ of the liposomes.

Furthermore this invention discloses a method to facilitate the induction of a CD8+ T cell response by the liposome/glycolipid adjuvant through modification of the lipid composition, thereby affecting gel-liquid crystalline phase transition of the liposomes. A preferred adjuvant disclosed by the invention is an adjuvant comprising liposomes consisting of an immunomodulator, cationic lipids and a neutral phospholipid changing the overall Tₘ of the liposomes.

The weight ratio between the cationic lipids and the neutral lipids are preferably between 19:1 (5% neutral lipid) and 4:16 (80% neutral lipid) and most preferably 12:8 (40% neutral lipid).

In addition to providing immunity to diseases, the adjuvant combinations of the present invention can also be used for producing antibodies against compounds which are poor immunogenic substances per se and such antibodies can be used for the detection and quantification of the compounds in question, e.g. in medicine and analytical chemistry.

### EXAMPLES

Material and Methods:

Vaccine Antigens

Ag85B-ESAT-6

The fusion protein of Ag85B and ESAT-6 (in the following designated Ag85B-ESAT-6) was produced as recombinant proteins as previously described (Olsen et al, 2001).

Influenza

Commercially available influenza split vaccine Begrivac was obtained from Novartis.

CtHl

CtHl is a fusion of the two Chlamydia antigens Ct521 and Ct443. The recombinant fusion protein was produced as follow: DNA fragments containing the genes of ct521 and ct443 were amplified from Ct serovar D genomic DNA by overlap extension PCR. Amplifications were carried out for 25 cycles each with denaturation at 94° C. for 30 sec, annealing at 55° C. for 30 sec, and extension at 72° C. for 2 min, using Phusion polymerase (Finnzymes, Espoo, Finland). Nucleotide sequencing was performed directly on the PCR products by MWG-Biotech AG (Germany) using specific sequencing primers. The ct521-ct443 gene fusion was created using the specific primer Ct521-fw-1 (5′-CAC CGG ATC CAT GTT AAT GCC TAA ACG AAC AAA ATT TC (SEQ ID NO: 1)) and Ct521-rev-1 (5′-CAC CCC GCT AGC AAA TAA ACT TAC CCT TTC CAC ACG CTT AAC AAA (SEQ ID NO: 2)) (ct521), Ct443-fw-1 (5′-TTT GTT AAG CGT GTG GAA AGG GTA AGT TTA TTT GCT AGC GGG GTG (SEQ ID NO: 3) and Ct443-rev-1 (5′-GGA TCC CTA ATA GAT GTG TGT ATT CTC TGT ATC AGA AAC TG (SEQ ID NO: 4)) (ct443) in a first round PCR using chlamydial DNA extracted as the template. The respective products were used as templates in second round PCR using the primers Ct521-fw1 and Ct443-rev-1. The resulting DNA fragment was cloned into pENTR/D-TOPO and subsequently into pDEST17 expression vector (Invitrogen, Copenhagen) thereby creating an in frame fusion with 6*His tag. The ct443-ct521 gene fusion was created analogous to CTH1 using the specific primer Ct443-fw-2 (5′-CAC CGG ATC CAG TTT ATT TGC TAG CGG GGT G (SEQ ID NO: 7)) and Ct443-rev-2 (5′-GAA ATT TTG TTC GTT TAG GCA TTA ACA TAT AGA TGT GTG TAT TCT CTG TAT CAG AAA CTG (SEQ ID NO: 13)) (ct443) and Ct521-fw-2 (5′-CAG TTT CTG ATA CAG AGA ATA CAC ACA TCT ATA TGT TAA TGC CTA AAC GAA CAA AAT TTC (SEQ ID NO: 5)) and Ct521-rev-2 (5′-GGA TCC CTA TAC CCT TTC CAC ACG CTT AAC AAA (SEQ ID NO: 6)) (ct521) in the first round PCR. The respective products were used as templates in second round PCR using the primers Ct443-fw-2 and Ct521-rev-2. The resulting DNA fragment was cloned into pENTR/D-TOPO (Invitrogen, Copenhagen) and subsequently into pDEST17 expression vector (Invitrogen, Copenhagen, Denmark).

The recombinant gene was expressed as purified as follows: E. coli BL-21 AI cells transformed with plasmid pDEST17 (Invitrogen, Copenhagen, Denmark) encoding both hybrids were grown at 37° C. to reach the logarithmic phase OD₆₀₀˜0.5 and protein expression was induced by adding arabinose to total concentration of 0.2%. The protein expression was induced for 4 hours and cells were harvested by centrifugation (6,000 g for 15 min.). E. coli were lysed using Bugbuster (Novagen, Darmstadt, Germany) containing Benzonase, rLysozyme and Protease inhibitor Cocktail I (Calbiochem, San Diego, Calif.) to avoid unwanted degradation. Lysis was performed at room temperature for 30 min. during gentle agitation. Inclusion bodies were isolated by centrifugation (10,000 g for 10 min.) The pellet was washed once with 1:5 diluted Bugbuster solution in 3M urea and then dissolved in 50 mM NaH₂PO₄, 0.4M NaCl, 8M Urea, 10% glycerol, 10 mM Imidazole pH 7.5. This solution was loaded onto a 5 ml HisTrap HP (Amersham Biosciences, Buckinghamshire, United Kingdom) and the bound proteins were eluted by applying a gradient of 50 to 500 mM imidazole. Fractions containing the desired recombinant protein were pooled, dialyzed against 20 mM ethanolamine, pH 9, 8M urea and applied to a 5 ml HiTrap Q Sepharose HP (Amersham Biosciences, Buckinghamshire, United Kingdom). The recombinant protein was eluted by applying a gradient of 0 to 1M NaCl over 10 column volumes. Analysis of all fractions was performed by SDS-PAGE. Protein concentrations were measured by the BCA protein assay (Pierce, Rockford, Ill., USA). The purity was assessed by SDS-PAGE followed by coomassie staining and western blot with anti-penta-His (Qiagen, Ballerup, Denmark) and anti-E. coli antibodies to detect contaminants (DAKO, Glostrup, Denmark). The two hybrid proteins were refolded by a stepwise removal of buffer containing urea ending up in 20 mM Citrate-phosphate buffer pH 4, 10% glycerol, 1 mM cysteine which yielded soluble hybrid protein. The purified hybrids were stored at −20° C. until use.

MSP1-19

The 19kdDa C-terminal protective fragment of MSP1 (Tian, Kumar et al. 1997) was amplified from Plasmodium yoelii genomic DNA using the PYMSP1-fw (5′-CAC CGG CAC ATA GCC TCA ATA GCT TTA AAC A (SEQ ID NO: 9)) and PYMSP1-rev (5′-CTA GCT GGA AGA ACT ACA GAA TAC ACC TT (SEQ ID NO: 10)) primers. Amplification was carried out for 25 cycles with denaturation at 94° C. for 30 sec, annealing at 55° C. for 30 sec, and extension at 72° C. for 2 min, using Phusion polymerase (Finnzymes, Espoo, Finland). The resulting DNA fragment was cloned into pENTR/D-TOPO (Invitrogen, Copenhagen) and subsequently into pDEST17 expression vector. The corresponding recombinant protein was purified by metal chelate affinity chromatography essentially as described (Theisen, Cox et al. 1994).

E7

The entire sequence of E7 antigen from Human Papilloma Virus strain 16 (HPV 16) was amplified from the murine tumor cell line TC-I (ATTC product no. CRL-2785) genomic DNA, using the oligonucleotides 5′-ggggacaagtttgtacaaaaaagcaggcttaATGCATGGAGATACACC TACATT-3′ (SEQ ID NO: 11) and 5′-ggggaccactttgtacaagaaagctgggtcTTATGGTTTCTGAGAACAGATGG (SEQ ID NO: 12). E7 gene specific sequences are in capital letters and the stop codon is underlined. Amplification was carried out for 30 cycles using the Iproof polymerase kit (Invitrogen, Copenhagen) with denaturation at 94° C. for 30 sec, annealing at 60° C. for 30 sec, and extension at 72° C. for 1 min. The resulting DNA fragment was inserted into the pDEST17 expression vector by two recombination steps as recommended by the producer (Invitrogen, Copenhagen). The vector encoded His tag was exploited to purify recombinant E7 protein from E. coli homogenate by a three-step procedure as previously described (Aagaard C. J. Dietrich, et al. Submitted).

Liposome Formulations

The DDA/TDB and DDA/DSPC/TDB liposomes were made using the thin lipid film method. Dimethyldioctadecylammonium Bromide (DDA, Mw=630.97) (Avanti Polar Lipids, Alabaster, Al), D-(+)-Trehalose 6,6′-dibehenate (TDB, Mw=987.5) (Avanti Polar Lipids, Alabaster, Al), 1,2-distearoyl-sn-Glycero-3-Phosphocholine (D(C18)PC=DSPC, Mw=791.16), 1,2-dibehenoyl-sn-Glycero-3-Phosphocholine (D(C22)PC, Mw=902.37) and 1,2-dilignoceroyl-sn-Glycero-3-Phosphocholine (D(C24)PC, Mw=958.48) (Avanti Polar Lipids, Alabaster, Al) were dissolved separately in chloroform methanol (9:1) to a concentration of 10 mg/ml. Specified volumes of each individual compound were mixed in glass test tubes. The solvent was evaporated using a gentle stream of N₂ and the lipid films were dried overnight under low pressure to remove trace amounts of solvent. The dried lipid films were hydrated in Tris-buffer (10 mM, pH=7.4) to the concentrations specified in Table 1, and placed on a 70° C. water bath for 20 min, the samples are vigorously shaken every 5 min.

TABLE 1
List a range of adjuvant formulation prepared in accordance with
the present invention.

| DDA/DSPC/TDB | Concentration | | |
|---|---|---|---|
| Ratio | DDA (mg/ml) | DSPC (mg/ml) | TDB (mg/ml) |
| 5/0/1 | 1.25 | 0 | 0.25 |
| 4/1/1 | 1.00 | 0.25 | 0.25 |
| 3/2/1 | 0.75 | 0.50 | 0.25 |
| 2/3/1 | 0.50 | 0.75 | 0.25 |
| 1/4/1 | 0.25 | 1.00 | 0.25 |

Animals

Female BALB/C or C57BL/6 mice, 8 to 12 weeks old, were obtained from or Harlan Scandinavia (Denmark).

Immunizations

Mice were immunized subcutaneously (s.c.) at the base of the tails up to three times with a two week interval between each immunization. The vaccines (0.2 ml/mice) consisted of 2 μg of the fusion protein Ag85B-ESAT-6, 1 μg of the influenza split vaccine, 5 μg of the CtHl, or 10 μg of MSP1-19 administered in 250 μg DDA and 50 μg of TDB or 150 μg DDA, 50 μg of TDB, 100 μg of DSPC. In some experiments, 500 μg/dose of aluminum hydroxide adjuvant (Alhydrogel 2%, Brenntag, Denmark) was included.

Immunization of female C57BL/6 mice with the HPV16 E7 antigen was done s.c. at the base of the tail at day 4, 7, 10 and 24 relative to the day of TC-1 tumor cell injection. Vaccines consisted of 5 μg of E7 administered in 150 μg DDA, 50 μg of TDB, 100 μg of DSPC. Mock vaccine consisted of saline mixed with 150 μg DDA, 50 μg of TDB, 100 μg of DSPC

Tumor Challenge

Female C57BL/6 mice were injected intradermally at the right flank with 5×10̂4 TC-1 cells (ATCC product no. CRL-2785) in 50 μl of phosphate buffered saline. Tumor growth was measured by palpation twice weekly, and mice were euthanized when the tumor reached a size of 200 mm².

Detection of Vaccine-Specific Antibodies by ELISA

Micro titers plates (Nunc Maxisorp, Roskilde, Denmark) were coated with influenza vaccine (1 μg/well), Ag85B-ESAT-6, CtHl, or MSP1-19 (all 0.5 μg/well) in PBS overnight at 4° C. Free binding sites were blocked with 2% skim milk in PBS. Individual mouse serum from three to six mice per group was analyzed in duplicate in fivefold dilutions at least 8 times in PBS containing bovine serum albumin starting with a 20-fold dilution. Horseradish peroxidase (HRP)-conjugated secondary antibodies (rabbit anti-mouse immunoglobulin G1; IgG1 and IgG2a/b/c; Zymed) diluted 1/2000 in PBS with 1% bovine serum albulin was added. After 1 h of incubation, antigen-specific antibodies were detected by TMB substrate as described by the manufacturer (Kem-En-Tec, Copenhagen, Denmark). In BALB/c mice, the IgG2a isotype was measured whereas IgG2b and c levels were analyzed in C57BL/6 mice, as the gene for IgG2a is deleted in this strain (Jouvin-Marche, Morgado et al. 1989).

Detection of CD8+Cells by FACS

Blood was collected by periorbital puncture and pooled groupwise. Peripheral blood mononuclear cells (PBMCs) were purified by centrifugation on Lympholyte cell separation media (Cedarlane Laboratories Ltd, Ontario, Canada) and washed in RPMI-1640 media (Invitrogen, Copenhagen, Denmark). Cells were restimulated with 5 μg/ml of recombinant E7 as described in Lindenstrom, Agger et al. 2009. Briefly, cells incubated for 1 hour at 37° C. with antigen and co-stimulatory antibodies (anti-CD28 and anti-CD49d, was then added Brefeldin A (10 μg/ml) and incubated a further 5 hours before cooling the cells to 4° C. and storing overnight. Cytokine producing T cells were stained using anti-IFN-γ-PE-Cy7, anti-TNF-α-PE, anti-CD4-APC-Cy7, anti-CD8-PerCp-Cy5.5, anti-CD44-FITC antibodies and flow cytometric analysis as described in Lindenstrom, Agger et al 2009.

### Example 1

### DSPC Incorporated in the Lipid Bilayer of DDA/TDB Vesicles Results in an Increased Tₘ

Lipid bilayers formed from DDA/TDB undergoes a characteristic gel to liquid crystal main phase transition with a main phase transition temperature Tₘ. The phase transition involves melting of the dialkyl chains in the vesicular bilayers, and the organization of the chains changes from a state characterized by a high degree of conformational order to state with a higher degree of disorder. A large transition enthalpy is associated with the chain melting process. This change in enthalpy is detected as a peak in the heat capacity curve with a maximum at the transition temperature, Tₘ. The transition temperature as well as the shape of the heat capacity curve depends on the nature of the polar head-group, the counter ion, and the length of the dialkyl chains. Generally the Tₘ values decreases with decreasing chain length and increasing asymmetry of the alkyl chains. The effect of an additional dialkyl surfactant on the thermotropic phase behavior can provide useful information on the interaction between the liposome components. Heat capacity curves were obtained using a VP-DSC differential scanning microcalorimeter (calorimetry Sciences Corp., Provo) of the power compensating type with a cell volume of 0.34 mL. Three consecutive upscans of 0.34 ml sample were performed at 30° C./h.

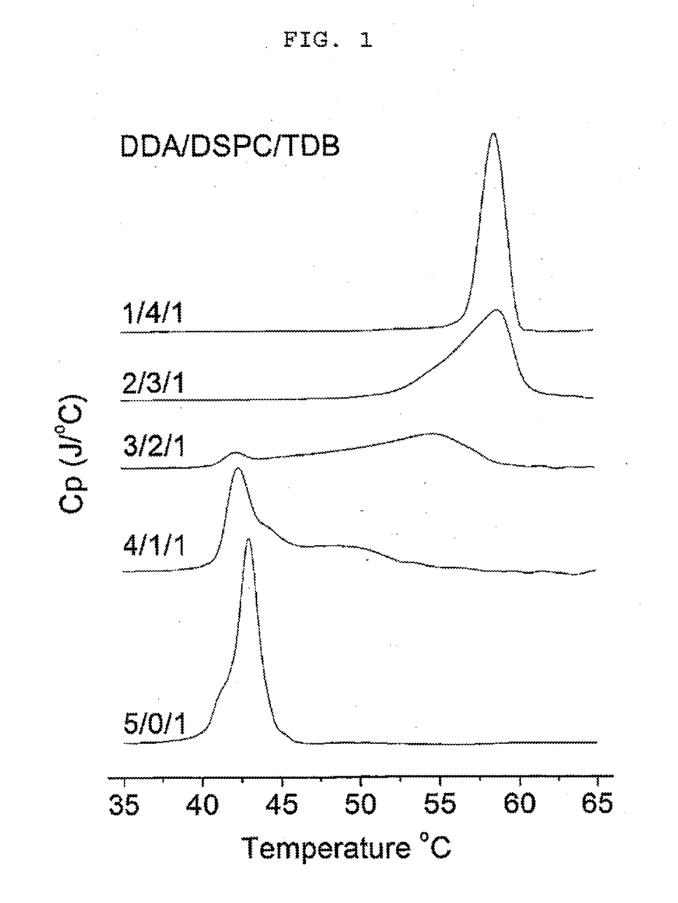

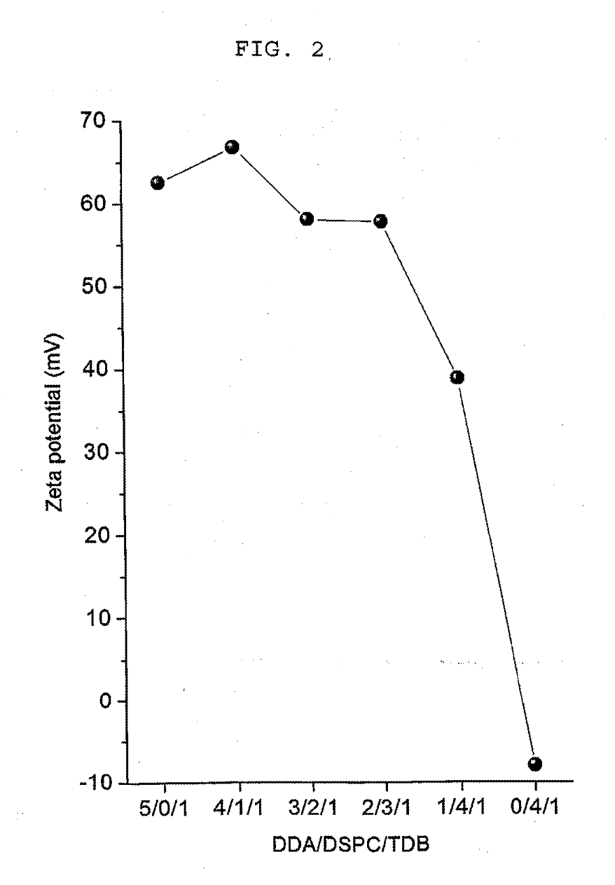

The DSC thermograms of the three component system consisting of DDA/DSPC/TDB shown in FIG. 1 demonstrate a marked influence of increasing the molar concentration of DSPC on the lipid-membrane thermodynamics. The membrane insertion of DSPC in the bilayers of the DDA/TDB liposomes is demonstrated by the increasing of the main phase transition temperature Tₘ. The gel to fluid transition of the DDA/TDB liposomes is characterized by a phase transition expanding from approx. 39 to 46° C. with Tₘ 43° C. The phase transition of the DDA/DSPC/TDB liposomes with the weight ratio 4/1/1 is broadened considerably and expands from 39 to approx. 55° C. This is most likely due to a small-scale compositional phase separation in the lipid membranes during the gel to fluid transition process. Replacement of more DDA with DSPC increases the phase transition temperature further and Tₘ of the DDA/DSPC/TDB liposomes with the weight ratio 1/4/1 is shifted upward about 16° C. above that of DDA/DSPC/TDB liposomes with the weight ratio 5/0/1.

### Example 2

### DSPC Incorporated in the Lipid Bilayer of DDA/TDB Vesicles Results in a Decreased Surface Charge

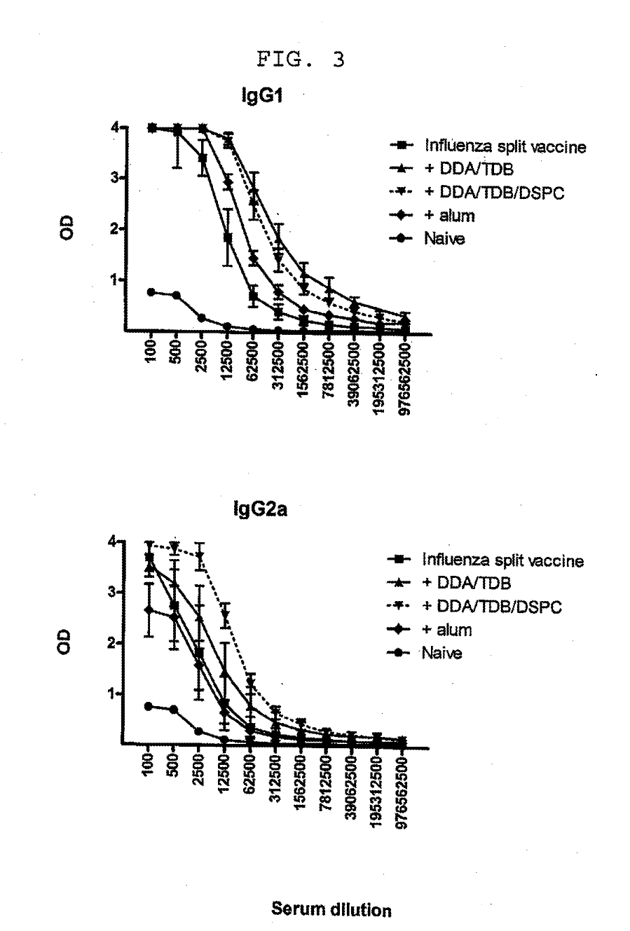

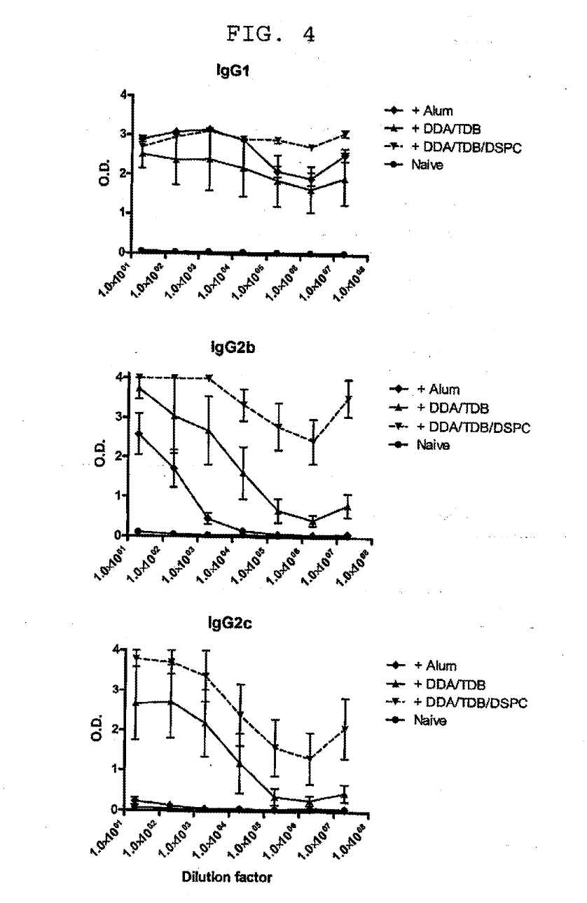

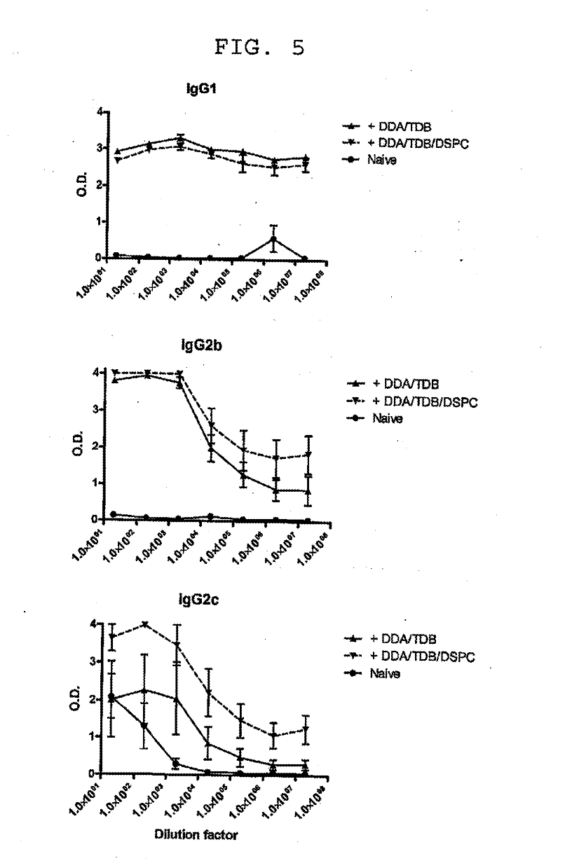

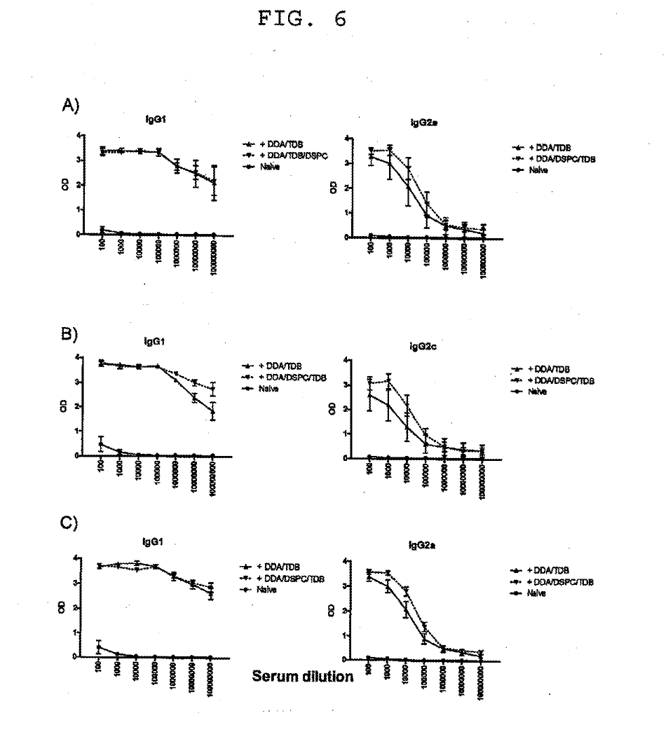

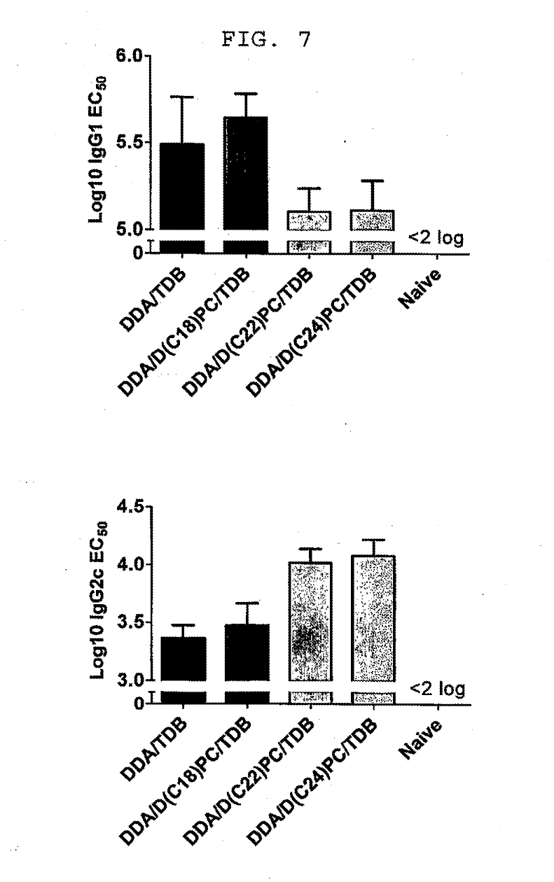

Replacement of DDA, being a strongly cationic quaternary ammonium compound, with DSPC being a zwitter-ionic surfactant with a neutral charge at pH=7.4, results in a decreased surface charge (FIG. 2). This was determined by the zeta-potential of the liposomes using a Malvern NanoZS (Malvern Instruments, Worcestershire, UK). However, only the replacement of more than 60% of the cationic surfactant resulted in a significant decrease in surface charge. That is DDA/DSPC/TDB with the weight ratio 5/0/1 had a zeta-potential of 62.5 mV and DDA/DSPC/TDB with the weight ratio 2/3/1 had a zeta-potential of 57.8 mV. Further replacement of DDA with DSPC to a DDA/DSPC/TDB ratio of 1/4/1 lead to a significant decrease of the zeta-potential to 38.9 mV.

### Example 3

### Induction of High Titers IgG2 Antibodies in Combination with Influenza Antigen

In order to analyze the antibody response obtained by using DDA/DSPC/TDB (ratio 3/2/1) as an adjuvant, groups of BALB/C mice were immunized with 1 μg of an influenza split vaccine in different preparations. In addition to DDA/DSPC/TDB, this also included an aluminum-hydroxide (alum) adjuvanted preparation, the DDA/TDB adjuvant as well as mice receiving the influenza vaccine without adjuvant. Four weeks after a single vaccination, mice were bled and the sera from individual mice analyzed for the generation of influenza vaccine-specific antibodies. As shown in FIG. 3, all tested adjuvants generated antigen-specific antibodies of the IgG1 isotypes at a level higher compared to mice receiving the vaccine without adjuvant. The highest levels were seen with DDA/DSPC/TDB and DDA/TDB. Analyzing the IgG2a response, no difference could be seen between the alum-adjuvanted group and the group of mice receiving the vaccine without adjuvant. The highest level of IgG2a was seen in the mice receiving influenza vaccine in DDA/DSPC/TDB.

### Example 4

### Induction of High Titers IgG2 Antibodies in Combination with Tuberculosis Vaccine Antigen

C57BL/6 mice were vaccinated three times with the tuberculosis vaccine candidate Ag85B-ESAT-6 in DDA/TDB, alum or DDA/DSPC/TDB (ratio 3/2/1). Three weeks after the last vaccination, mice were bled and the Ag85B-ESAT-6-specific antibodies assessed in the serum by ELISA. The levels of IgG1 antibodies were comparable in all three groups as shown in FIG. 3. In contrast, levels of IgG2 bas well as IgG2c were higher in the mice receiving Ag85B-ESAT-6 in DDA/DSPC/TDB.

### Example 5

### Induction of High Titers IgG2 Antibodies in Combination with Malaria Antigen

C57BL/6 mice were vaccinated three times with the malaria protein MSP1-19 in either DDA/TDB or DDA/DSPC/TDB (ratio 3/2/1). Three weeks after the last vaccination, mice were bled and the MSPI-19-specific antibodies assessed in the serum by ELISA. Again, the levels of

IgG1 antibodies were comparable in all three groups (FIG. 5) whereas levels of IgG2b as well as IgG2c were higher in the mice vaccinated with MSP1-19 in DDA/DSPC/TDB.

### Example 6

### Induction of High Titers IgG2 Antibodies in Combination with Chlamydia Antigen

Different mouse strains (BALB/C, C57BL/6, BALB/c×C57BL/6 F₁ mice were vaccinated three times with the chlamydia fusion antigen CtHl in either DDA/TDB or DDA/DSPC/TDB and the presence of CtHl-specific antibodies analyzed three weeks after the final vaccination. In all three mouse strains, IgG1 levels were comparable between DDA/TDB and DDA/DSPC/TDB whereas the amount of IgG2 antibodies was higher in the mice receiving CtHl in DDA/DSPC/TDB.

### Example 7

### Induction of Increased IgG2 and Decreased IgG1 Antibody Titers by Further Increasing the Gel-to-Liquid Phase Transition Temperature

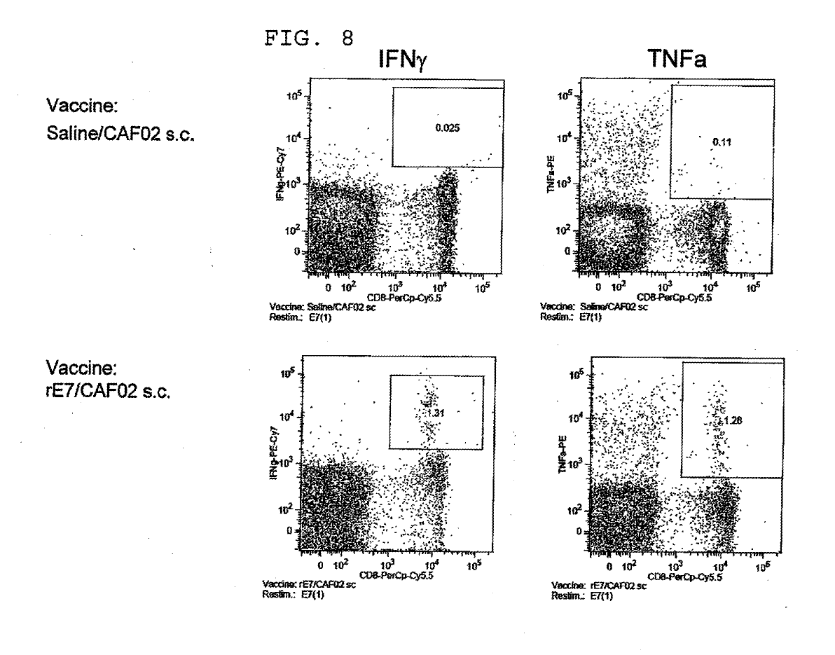

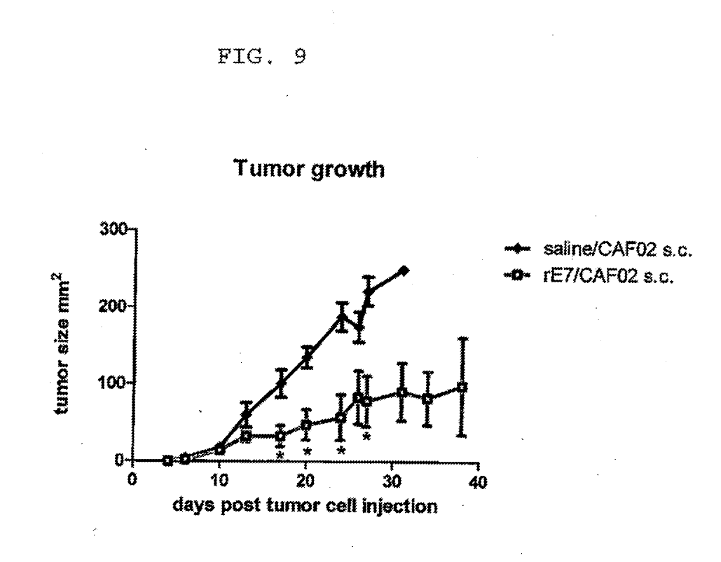

C57BL/6 mice were vaccinated three times with the tuberculosis vaccine candidate Ag85B-ESAT-6 in DDA/TDB, DDA/D(C18)PC/TDB (w. ratio 3/2/1, D(C18)PC=DSPC), DDA/D(C22)PC/TDB (w. ratio 3/2/1) or DDA/D(C18)PC/TDB (w. ratio 3/211). Three weeks after the last vaccination, mice were bled and the Ag85B-ESAT-6-specific antibodies assessed in the serum by ELISA. The levels of IgG1 antibodies were reduced in the groups containing the C22 and C24 PC's as shown in FIG. 7. In contrast, levels of IgG2c were higher in the same two groups. As the gel-to-liquid phase transition shifts towards higher temperatures with longer chain-lengths this supports the theory that higher phase transition temperatures shift the humoral immune response towards more CMI mediated antibody production.

### Example 8

### Induction of a CD8+ T Cell Response Using DDA/DSPC/TDB Lipids

Female C57BL/6 mice were vaccinated four times with the Human Papilloma virus antigen E7 in DDA/DSPC/TDB (w. ratio 3/2/1), at days 4, 7, 10 and 24 relative to day of challenge with the tumor cell line TC-1. Eight days after third vaccination, mice were bled by periorbital puncture and the number of E7 antigen specific CD8+ T cells was assessed by antigen restimulation of peripheral blood mononuclear cells (PBMCs) and flow cytometric analysis of cells producing cytokines in response to antigen recognition.

### Example 9

### DSPC Incorporated in the Lipid Bilayer of DDA/TDB Vesicles Results in an Increased Average Particle Size But Not in Destabilization of the Particles

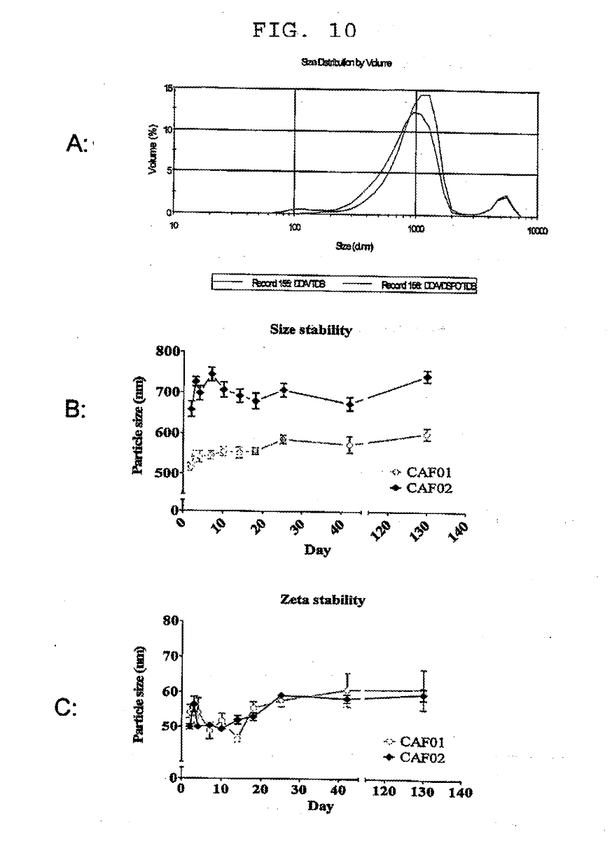

The long term particle size and charge stability of formulations containing DDA/TDB (w/w ratio=5:1) and DDA/DSPC/TDB (w/w ratio=3:2:1) were analyzed using dynamic light scattering. Data showed that the average particle size was increased with approximately 150 nm but that the poly dispersity were similar (FIG. 10A). Furthermore both the average particle size (FIG. 10B) and surface charge (FIG. 10C) were maintained over a period of at least 3 months.

### REFERENCES

Aagaard, C., J. Dietrich, et al. (Submitted), “Multistage TB vaccine induces long term protection and polyfunctional immune responses”.
Ahlborg, N., I. T. Ling, et al. (2000). “Linkage of exogenous T-cell epitopes to the 19-kilodalton region of Plasmodium yoelii merozoite surface protein 1 (MSP1(19)) can enhance protective immunity against malaria and modulate the immunoglobulin subclass response to MSP1(19).” Infect Immun 68(4): 2102-9.
Bakouche, O. and D. Gerlier (1986). “Enhancement of immunogenicity of tumour virus antigen by liposomes: the effect of lipid composition.” Immunology 58(3): 507-13.
Campbell, R. B., E. B. Brown, et al. (2002). “Drug Delivery Formulations and Targeting.” WO 02/03959.
Davidsen, J., I. Rosenkrands, et al. (2006). “Compositions and Methods for Stabilizing Lipid based Adjuvant formulations using Glycolipids.” W02005DK00467 20050705.
Davidsen, J., I. Rosenkrands, et al. (2005). “Characterization of cationic liposomes based on dimethyldioctadecylammonium and synthetic cord factor from M. tuberculosis (trehalose 6,6′-dibehenate)-a novel adjuvant inducing both strong CMI and antibody responses.” Biochim Biophys Acta 1718(1-2): 22-31.
Gall, D. (1966). “The adjuvant activity of aliphatic nitrogenous bases.” Immunology 11(4): 369-86.
Gluck, R. (1995). “Liposomal presentation of antigens for human vaccines.” Pharm Bio-technol. 6: 325-345.
Gregoriadis, G. (1995). “Engineering liposomes for drug delivery: progress and problems.” Trends Biotechnol 13(12): 527-37.
Harboe, M., A. S. Malin, et al. (1998). “B-cell epitopes and quantification of the ESAT-6 protein of Mycobacterium tuberculosis.” Infect Immun 66(2): 717-23.
Jouvin-Marche, E., M. G. Morgado, et al. (1989). “The mouse Igh-la and Igh-lb H chain constant regions are derived from two distinct isotypic genes.” Immunogenetics 29(2): 92-7.
Lindenstrom, T., E. M. Agger, et al. (2009). “Tuberculosis subunit vaccination provides long-term protective immunity characterized by multifunctional CD4 memory T cells.” J Immunol 182(12): 8047-55.
Liu, D., F. Liu, et al. (1997). “Emulsion and Micellar Formulations for the Delivery of Biologically Active Substances to Cells.” WO 97111682.
McMichael, A. J., F. Gotch, et al. (1981). “The human cytotoxic T cell response to influenza A vaccination.” Clin Exp Immunol 43(2): 276-84.
Moingeon, P., J. Haensler, et al. (2001). “Towards the rational design of Th1 adjuvants.” Vaccine 19(31): 4363-4372.
Moore, T., C. O. Ekworomadu, et al. (2003). “Fe receptor-mediated antibody regulation of T cell immunity against intracellular pathogens.” J Infect Dis 188(4): 617-24.
Nimmerjahn, F., P. Bruhns, et al. (2005). “FcgammaRIV: a novel FeR with distinct IgG subclass specificity.” Immunity 23(1): 41-51.
Regnault, A., D. Lankar, et al. (1999). “Fcgamma receptor-mediated induction of dendritic cell maturation and major histocompatibility complex class I-restricted antigen presentation after immune complex internalization.” J Exp Med 189(2): 371-80.
Stanfield, J. P., D. Gall, et al. (1973). “Single-dose antenatal tetanus immunisation.” The Lancet 301(7797): 215-19.
Taylor, R. R., S. J. Allen, et al. (1998). “IgG3 antibodies to Plasmodium falciparum merozoite surface protein 2 (MSP2): increasing prevalence with age and association with clinical immunity to malaria.” Am J Trop Med Hyg 58(4): 406-13.
Theisen, M., G. Cox, et al. (1994). “Immunogenicity of the Plasmodium falciparum glutamate-rich protein expressed by vaccinia virus.” Infect Immun 62(8): 3270-5.
Tian, J. H., S. Kumar, et al. (1997). “Comparison of protection induced by immunization with recombinant proteins from different regions of merozoite surface protein 1 of Plasmodium yoelii.” Infect Immun 65(8): 3032-6.
Zaks, K., M. Jordan, et al. (2006). “Efficient immunization and cross-priming by vaccine adjuvants containing TLR3 or TLR9 agonists complexed to cationic liposomes.” J Immunol 176(12): 7335-45.

1. A method for modifying the gel-liquid crystalline phase transition temperature (Tm) of the cationic liposomes of adjuvants comprising cationic liposomes stabilized with glycolipids by incorporating 1-Acyl-2-Acyl-sn-Glycero-3-Phosphocholine (DxPC), wherein 1-Acyl and 2-Acyl each is independently a long chain fatty acid containing from 12 to 24 carbon (C) atoms.

2. The method according to claim 1 where the cationic liposomes comprise dimethyldidodecanoylammonium, dimethylditetradecylammonium, dimethyldihexadecylammonium, DDA, DODA, DOTAP, 1,2-dimyristoyl-3-trimethylammonium-propane, 1,2-dipalmitoyl-3-trimethylammonium-propane, 1,2-distearoyl-3-trimethylammonium-propane, DODAP, DOTMA, DMTAP, DPTAP or DSTAP.

3. The method according to claim 1 where the glycolipids are TDB or MMG.

4. The method according to claim 3, where the fatty acids are lauric (12C), myristic (14C), palmitic (16C), stearic (18C), arachidonic (20C), Behenic (22C) or lignoceric (24C) acid.

5. The method according to claim 1 where the weight ratio between the cationic lipids and the DxPC neutral lipids is between 19:1 (5% neutral lipid) and 4:16 (80% neutral lipid).

6. An adjuvant prepared according the method according to claim 1.

7. An adjuvant according to claim 6 additionally comprising an immunomodulator.

8. An adjuvant according to claim 7 where the immunomodulator is a TLR ligand, polyinosinic polycytidylic acid (poly-IC) or derivatives thereof, TDM or derivatives thereof, MMG or derivatives thereof, zymosan, tamoxifen, CpG oligodeoxynucleotides, double-stranded RNA (dsRNA), or muramyl dipeptide (MDP) or analogs thereof.

9. A vaccine comprising the adjuvant according to claim 6.

10. A vaccine according to claim 9 comprising an antigen.

11. The vaccine according to claim 10, wherein said antigen is a tuberculosis, malaria, Chlamydia, influenza, HPV, HIV or cancer antigen.

12. The method according to claim 5 where the weight ratio between the cationic lipids and the DxPC neutral lipids is 12:8 (40% neutral lipid).

13. The adjuvant according to claim 8, wherein said TLR ligand is MPL (monophosphoryl lipid A) or a derivative thereof.

14. The adjuvant according to claim 8, wherein the derivative of TDM is TDB.


SEQUENCE LISTING (13 sequences)

SEQ ID NO: 1 - DNA, 38 bp, Chlamydia trachomatis
caccggatccatgttaatgcctaaacgaacaaaatttc

SEQ ID NO: 2 - DNA, 45 bp, Chlamydia trachomatis
caccccgctagcaaataaacttaccctttccacacgcttaacaaa

SEQ ID NO: 3 - DNA, 45 bp, Chlamydia trachomatis
tttgttaagcgtgtggaaagggtaagtttatttgctagcggggtg

SEQ ID NO: 4 - DNA, 41 bp, Chlamydia trachomatis
ggatccctaatagatgtgtgtattctctgtatcagaaactg

SEQ ID NO: 5 - DNA, 60 bp, Chlamydia trachomatis
cagtttctgatacagagaatacacacatctatatgttaatgcctaaacgaacaaaatttc

SEQ ID NO: 6 - DNA, 33 bp, Chlamydia trachomatis
ggatccctataccctttccacacgcttaacaaa

SEQ ID NO: 7 - DNA, 31 bp, Chlamydia trachomatis
caccggatccagtttatttgctagcggggtg

SEQ ID NO: 8 - DNA, 57 bp, Chlamydia trachomatis
attttgttcgtttaggcattaacatatagatgtgtgtattctctgtatcagaaactg

SEQ ID NO: 9 - DNA, 31 bp, Plasmodium yoelii
caccggcacatagcctcaatagctttaaaca

SEQ ID NO: 10 - DNA, 29 bp, Plasmodium yoelii
ctagctggaagaactacagaatacacctt

SEQ ID NO: 11 - DNA, 54 bp, Human papillomavirus type 16
ggggacaagtttgtacaaaaaagcaggcttaatgcatggagatacacctacatt

SEQ ID NO: 12 - DNA, 53 bp, Human papillomavirus type 16
ggggaccactttgtacaagaaagctgggtcttatggtttctgagaacagatgg

SEQ ID NO: 13 - DNA, 60 bp, Chlamydia trachomatis
gaaattttgttcgtttaggcattaacatatagatgtgtgtattctctgtatcagaaactg